(19) United States
(12) Patent Application Publication
(10) Pub. No.: US 2023/0240759 A1
(43) Pub. Date: Aug. 3, 2023

(54) MODULAR AND DEPTH-SENSING SURGICAL HANDPIECE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Rahul Khare, Sewickley, PA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/099,414

(22) Filed: Jan. 20, 2023

Related U.S. Application Data
(60) Provisional application No. 63/305,026, filed on Jan. 31, 2022.

(51) Int. Cl. A61B 34/20; A61B 17/16; A61B 34/00

(52) U.S. Cl. CPC A61B 34/20; A61B 17/1659; A61B 17/162; A61B 34/25; A61B 17/1622; A61B 2017/00725

(57) ABSTRACT

Disclosed herein are methods, systems, and apparatuses related to a computer-assisted surgical system including various hardware and software components working together to enhance surgical workflows. The disclosed techniques may be applied to, for example, shoulder, hip, and knee arthroplasties, as well as other surgical interventions. In one example, a device including a powered handheld device, a fixation mechanism, removably attached to the handheld device, and a surgical tool held by the fixation mechanism, wherein one or more combination surgical tools/fixation mechanisms are preoperatively attached to the handheld device and calibrated. In another example, a method includes for each of one or more surgical tools, placing the surgical tool in a fixation mechanism, attaching the fixation mechanism to a handheld device to create a surgical tool/fixation mechanism combination, calibrating the handheld device with the fixation mechanism and surgical tool, and storing the calibration information for the tool.

### RELATED APPLICATIONS

This application claims the benefit of U.S. Provisional Patent Application No. 63/305,026, filed Jan. 31, 2022, entitled “MODULAR AND DEPTH SENSING SURGICAL HANDPIECE”, the contents of which are incorporated herein in their entirety.

### TECHNICAL FIELD

The present disclosure relates generally to methods, systems, and apparatuses related to a computer-assisted surgical system that includes various hardware and software components that work together to enhance surgical workflows. The disclosed techniques may be applied to, for example, shoulder, hip, and knee arthroplasties, as well as other surgical interventions such as arthroscopic procedures, spinal procedures, maxillofacial procedures, rotator cuff procedures, ligament repair and replacement procedures.

### BACKGROUND OF THE PRESENT DISCLOSURE

Surgical navigation for primary joint replacement, for example, total knee arthroplasty (TKA) or total hip arthroplasty (THA) has been in use for the last 20 years. Surgical navigation technology can be used to guide or assist the surgeon's movements during an operation. It can track and display the real-time position of each surgical instrument and anatomical structures in the surgical field. The position of surgical instruments can be visualized by projection of the instrument onto a model of the patient's anatomy.

Surgical navigation technology is often embodied in a computer-assisted surgical system (CASS) of the type shown in FIG. 1. Such systems often include an end effector, shown as reference 105B in FIG. 1. End effector 105B may be, for example, a handheld device 105B or instrument used by the surgeon that holds a surgical tool or instrument during its use. One example of such a handheld device 105B is depicted in FIG. 2. The position and orientation of the handheld device 105B and, more particularly, the position of the tip of the tool 206 held by the handheld device, can be tracked using, for example, an array of fiducial markers 202 attached to the handheld device 105B and tracked by one or more cameras in the surgical theater.

In a TKA procedure, the surgical tool 206 may be, for example, a burr which is used by the surgeon to remove portions of a bone or to shape a bone in preparation for accepting an implant. When the burr is placed in the handheld device 105B, the device must undergo a calibration procedure to precisely determine the relative position of the tip of the burr with respect to the fiducial array 202, such that the position of the tip of the burr can be positively tracked during the procedure. Often, such surgical procedures require different sizes of burrs at various stages of the procedure. Currently, each time a new burr is placed in the handheld device 105B, the surgeon must suspend the procedure and perform the calibration.

Therefore, it would be desirable to provide a way for the surgeon to eliminate the calibration step each time a new tool is placed in the handheld device.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure illustrates two examples of the present disclosure which eliminate the need for intra-operative recalibration each time a new tool is placed in the handheld device.

In a first example, the handheld device 105B is provided with one or more removable tool fixation mechanisms (e.g., a “chuck”) 204. The fixation mechanism 204 can be attached to the handheld device 105B with a high degree of precision. One or more tools 206 to be used during the surgical procedure are each inserted into a fixation mechanism 204 and the handheld device 105B is calibrated for each tool pre-operatively. System software within the CASS may track the one or more tools 206 and record their positions with respect to their respective fixation mechanisms 204. As such, when attached to the handheld device 105B, with position of the tip of the tool with respect to the fiducial array 202, and thus with respect to the rest of the surgical field, is known. Intraoperatively, to replace the tool, the fixation mechanism 204 containing tool 206 is removed from the handheld device 105B and the fixation mechanism 204 containing the new tool 206 is attached to the handheld device 105B. The system software is then informed of the change and is able to load the calibration information for the newly-installed tool.

In a second example of the present disclosure, the fixation mechanism 204 is provided with a sensor internal to the fixation mechanism 204 that is able to track the position of the shaft of the tool 206 as it is inserted into the fixation mechanism 204. When the surgeon inserts a new tool 206 into the fixation mechanism 204, the system software of the CASS will track the position of the shaft of the tool 206 as it is inserted and will inform the surgeon when the tool 206 has been inserted to the proper depth with respect to the fixation mechanism 204.

In a first example of the present disclosure, a method comprises, for each of one or more surgical tools, placing the surgical tool in a fixation mechanism, attaching the fixation mechanism to a handheld device, calibrating the handheld device with the fixation mechanism and surgical tool and storing the calibration information for the tool.

In the first example, or any other example disclosed herein, the method further comprises wherein the step of calibrating comprises touching the surgical tool to one or more predefined spots such that a position of fiducial markers attached to the handheld device may be read by a tracking system.

In the first example, or any other example disclosed herein, a system comprises a processor and software that, when executed by the processor, performs the method of the first example.

In the first example, or any other example disclosed herein, the software further causes the system to provide instructions to the user indicating the exact steps required to calibrate each surgical tool.

In the first example, or any other example disclosed herein, the software further causes the system to receive an interoperative request to change the surgical tool and retrieve and load calibration information for the requested surgical tool.

In the first example, or any other example disclosed herein, the software further causes the system to instruct the user to connect the requested surgical tool and fixation mechanism to the handheld device.

In the first example, or any other example disclosed herein, the software further causes the system, in a preoperative phase, to receive an identification of one or more surgical tools to be used during a surgical procedure, determine a calibration procedure for the identified surgical tools and calibrate each identified surgical tool using the identified procedures.

In a second example of the present disclosure, a device comprises a powered handheld device, a fixation mechanism, removably attached to the handheld device and a surgical tool held by the fixation mechanism, wherein one or more combination surgical tools and fixation mechanisms are preoperatively attached to the handheld device and calibrated.

In the second example, or any other example disclosed herein, the device further comprises wherein the combination surgical tool and fixation mechanism attached to the powered handheld device may be dynamically changed during a surgical procedure by disconnecting a first fixation mechanism containing a first surgical tool and connecting a second fixation mechanism containing a second surgical tool to the powered handheld device.

In a third example of the present disclosure, a device comprises a powered handheld device, a fixation mechanism, removably attached to the handheld device, a surgical tool held by the fixation mechanism and a sensor for sensing an extent to which a shaft of the surgical tool has been inserted into the fixation mechanism.

In the third example, or any other example disclosed herein, the device further comprises wherein the sensor is an optical sensor and further wherein the shaft of the surgical tool has markings thereon detectable by the optical sensor.

In the third example, or any other example disclosed herein, the device further comprises wherein the sensor is a pressure sensor sensing pressure exerted by the shaft of the surgical tool as it is inserted into the fixation mechanism.

In the third example, or any other example disclosed herein, a system comprises a processor and software that, when executed by the processor, causes the system to receive an interoperative request to change the surgical tool and retrieve and load calibration information for the requested surgical tool.

In the third example, or any other example disclosed herein, the software further causes the system to receive preoperative identification of all surgical tools to be used during a surgical procedure and store calibration parameters associated with the identified surgical tools.

In the third example, or any other example disclosed herein, the software further causes the system to receive an interoperative request to change the surgical tool connected to the powered handheld device, retrieve and load calibration parameters for the selected surgical tool and provide feedback to a user of the system indicating proper positioning of the shaft of the surgical tool with respect to the fixation mechanism, based on readings from the sensor.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

The accompanying drawings, which are incorporated in and form a part of the specification, illustrate the examples of the present disclosure and together with the written description serve to explain the principles, characteristics, and features of the present disclosure. In the drawings:

FIG. 1 depicts an operating theatre including an illustrative computer-assisted surgical system (CASS) in accordance with described examples of the present disclosure.

FIG. 2 depicts an exemplary handheld device of the type with which the present disclosure may be implemented.

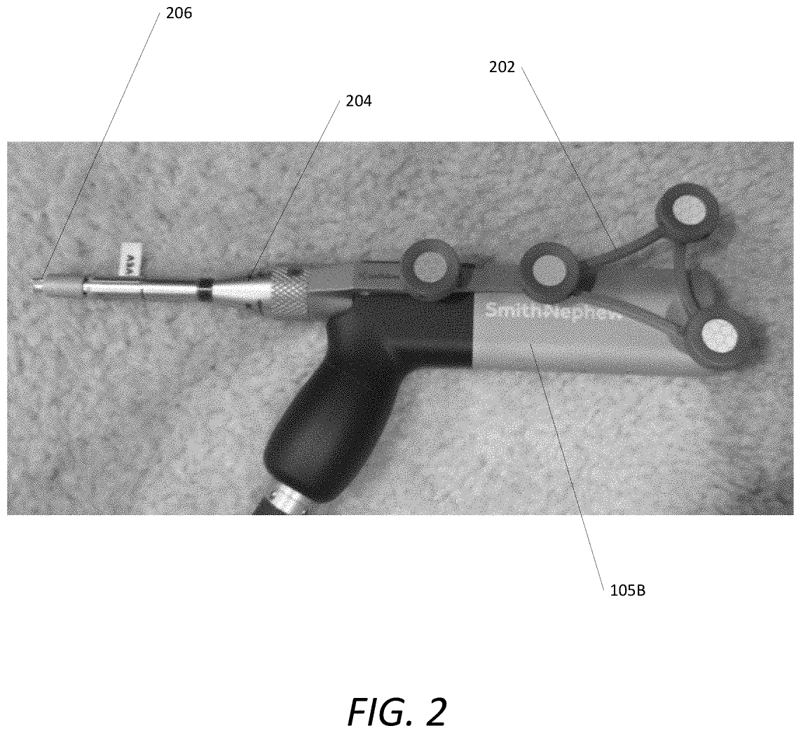

FIG. 3 depicts a first example of the present disclosure in which the handheld device is configured to accept a modular fixation element.

FIG. 4 is a flowchart showing the process flow of the system software of a CASS utilizing a first example of the present disclosure.

FIG. 5 depicts a first variation of the second example in which the fixation element is provided with an optical sensor.

FIG. 6 depicts a second variation of the second example of the present disclosure in which the fixation element is provided with a pressure sensor.

FIG. 7 is a flowchart showing the process flow the system software of a CASS as utilizing the second example of the present disclosure.

### DEFINITIONS

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon could also apply, in some examples to a technician or nurse.

### DETAILED DESCRIPTION

This disclosure is not limited to the particular systems, devices and methods described, as these may vary. The terminology used in the description is for the purpose of describing the particular versions or examples only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

### CASS Overview

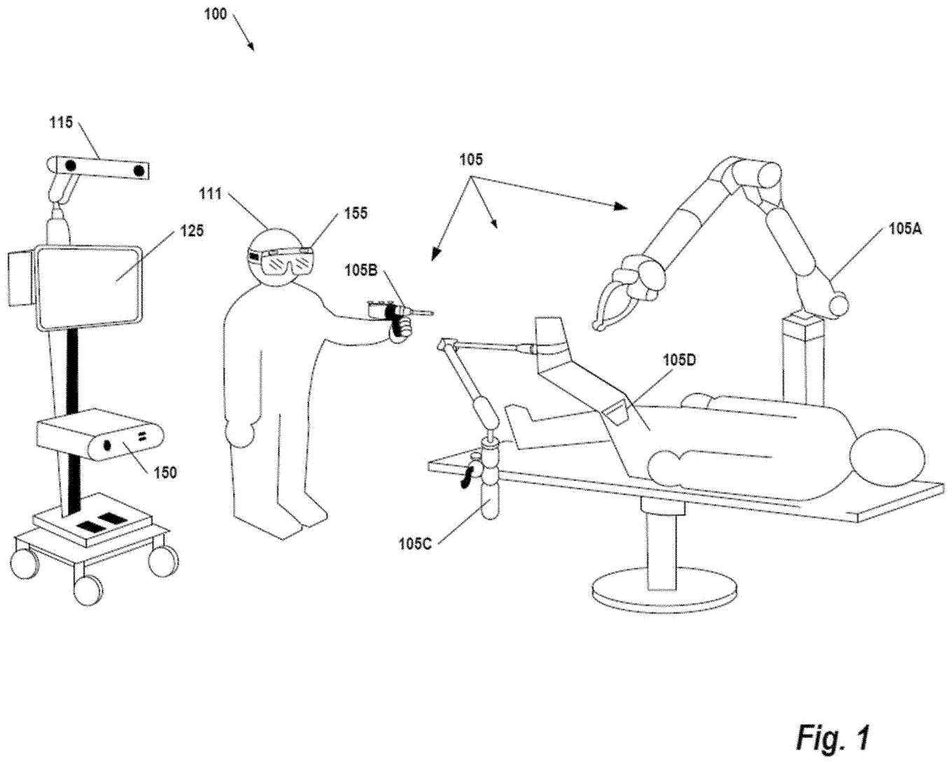

FIG. 1 provides an illustration of an example computer-assisted surgical system (CASS) 100, according to some examples. As described in further detail in the sections that follow, the CASS uses computers, robotics, and imaging technology to aid surgeons in performing orthopedic surgery procedures such as total knee arthroplasty (TKA) or total hip arthroplasty (THA). For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 105 positions surgical tools relative to a patient during surgery. The exact components of the effector platform 105 will vary, depending on the example employed. For example, for a knee surgery, the effector platform 105 may include an end effector 105B that holds surgical tools or instruments during their use. The end effector 105B may be a handheld device or instrument used by the surgeon (e.g., a NAVIO® hand piece or a cutting guide or jig) or, alternatively, the end effector 105B can include a device or instrument held or positioned by a robotic arm 105A.

The effector platform 105 can include a limb positioner 105C for positioning the patient's limbs during surgery. One example of a limb positioner 105C is the SMITH AND NEPHEW SPIDER2 system. The limb positioner 105C may be operated manually by the surgeon or alternatively change limb positions based on instructions received from the surgical computer 150 (described below).

The effector platform 105 can also include a cutting guide or jig 105D that is used to guide saws or drills used to resect tissue during surgery. Such cutting guides 105D can be formed integrally as part of the effector platform 105 or robotic arm 105A or cutting guides can be separate structures that can be matingly and/or removably attached to the effector platform 105 or robotic arm 105A. The effector platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The tracking system 115 uses one or more sensors to collect real-time position data that locates the patient's anatomy and surgical instruments. For example, for TKA procedures, the tracking system may provide a location and orientation of the end effector 105B during the procedure. In addition to positional data, data from the tracking system 115 can also be used to infer velocity/acceleration of anatomy/instrumentation, which can be used for tool control. In some examples, the tracking system 115 may use a tracker array attached to the end effector 105B to determine the location and orientation of the end effector 105B. The position of the end effector 105B may be inferred based on the position and orientation of the tracking system 115 and a known relationship in three-dimensional space between the tracking system 115 and the end effector 105B. Various types of tracking systems may be used in various examples of the present disclosure including, without limitation, Infrared (IR) tracking systems, electromagnetic (EM) tracking systems, video or image based tracking systems, and ultrasound registration and tracking systems.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some examples, this can include stereoscopic, tri-scopic, quad-scopic, etc. imaging. In addition to the camera array, which in some examples is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system.

In some examples, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some examples, certain markers, such as fiducial markers that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one or two dimensional optical codes (barcode, QR code, etc.) can be affixed to objects in the theater to provide passive identification that can occur based on image analysis. If these codes are placed asymmetrically on an object, they can also be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some examples, augmented reality headsets can be worn by surgeons and other staff to provide additional camera angles and tracking capabilities.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient can also involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process can also include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

The display 125 provides graphical user interfaces (GUIs) that display images and other information relevant to the surgery. For example, in one example, the display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 125 may include, for example, one or more computer monitors. As an alternative or supplement to the display 125, one or more members of the surgical staff may wear an augmented reality (AR) head mounted device (HMD). For example, in FIG. 1 the surgeon 111 is wearing an AR HMD 155 that may, for example, overlay pre-operative image data on the patient or provide surgical planning suggestions. Various example uses of the AR HMD 155 in surgical procedures are detailed in the sections that follow.

Surgical computer 150 provides control instructions to various components of the CASS 100, collects data from those components, and provides general processing for various data needed during surgery. In some examples, the surgical computer 150 is a general purpose computer. In other examples, the Surgical Computer 150 may be a parallel computing platform that uses multiple central processing units (CPUs) or graphics processing units (GPU) to perform processing. In some examples, the Surgical computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

In some examples, CASS 100 may include a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some examples, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In examples in which an end effector 105B is used, the end effector may be positioned at the end of the robotic arm 105A such that any motor control operations are performed within the robotic arm system. In examples in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some examples, the robotic arm 105A can be moved by hand (e.g., by the surgeon) or with internal motors into its ideal position and orientation for the task being performed. In some examples, the robotic arm 105A may be enabled to operate in a “free” mode that allows the surgeon to position the arm into a desired position without being restricted. While in the free mode, the position and orientation of the robotic arm 105A may still be tracked as described above. In one example, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 can also place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other examples, movement of the robotic arm 105A or robotically controlled end effector 105B can be controlled entirely by the CASS 100 without any, or with only minimal, assistance or input from a surgeon or other medical professional. In still other examples, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

### Description of Examples of the Present Disclosure

While various illustrative examples incorporating the principles of the present disclosure have been disclosed, the present disclosure is not limited to the disclosed examples. Instead, this disclosure is intended to cover any variations, uses, or adaptations of the present disclosure and use its general principles. Further, this application is intended to cover such departures from the present disclosure that are within known or customary practice in the art to which these disclosures pertain.

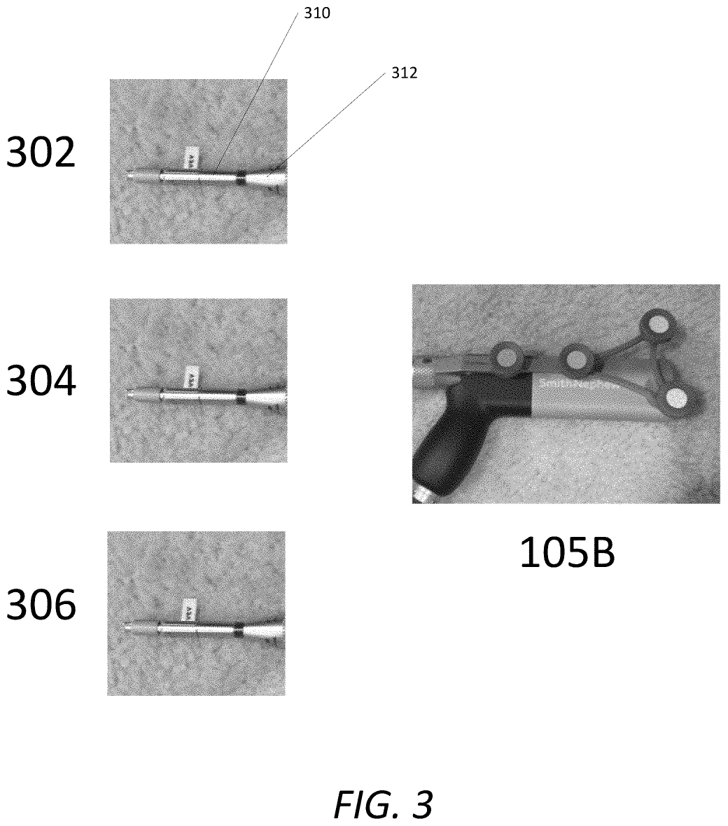

In a first example of the present disclosure, the current design of the handheld device is modified to accept a modular fixation mechanism 312 which may be mated with the handheld device 105B with a high degree of precision. The fixation mechanism 312, along with the burr (or other type of tool) 310 is provided as separate modules, for example, 302, 304, 306 as shown in FIG. 3. During system initialization, all of the prepared modules (i.e., tool+fixation mechanism) that need to be used during the procedure can be calibrated using a calibration procedure built into the software. Thus, during the bone cutting stage of the surgical procedure, the tools 310 can be freely changed without any need for re-calibration simply by attaching the required tool/fixation mechanism combination to the handheld device.

FIG. 3 shows an exemplary example in which three exemplary modules 302, 304, 306 have been prepared in advance for the surgical procedure. Each module comprises a tool 310 and a fixation mechanism or chuck 312. The tools 310 to be used during the surgical procedure may be selected in advanced and prepared by inserting each tool 310 into a fixation mechanism 312 to create the modules. During system initialization, each module can be attached to the handheld device 105B and calibrated, and the calibration information stored by the software of CASS 100. During the surgical procedure, when the surgeon wishes to switch tools, the surgeon indicates to the software which of the three modules 302, 304, 306 is being attached to handheld device 105B and the software then loads the calibration information for the selected module. As would be realized by one of skill in the art, any number of modules maybe pre-operatively prepared and calibrated for the surgical procedure.

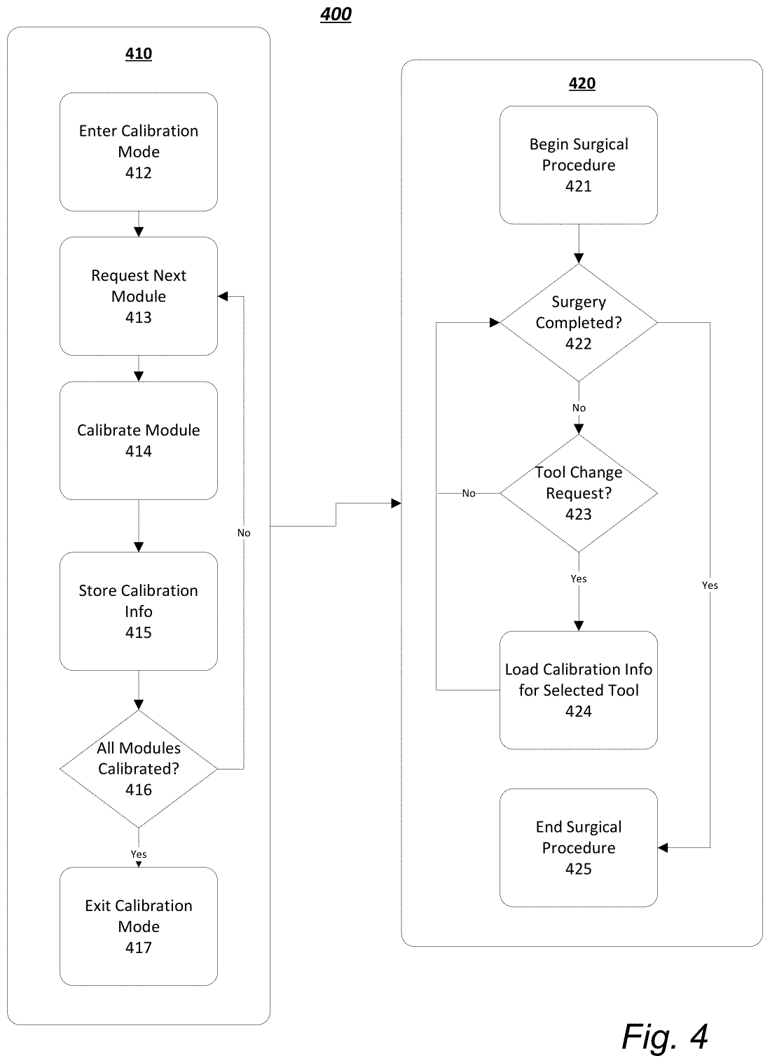

FIG. 4 is a flowchart showing process 400 implemented as software within CASS 100 for supporting the first example of the present disclosure. The process is divided into two phases, a pre-operative phase 410 and an intra-operative phase 420. In preferred examples, the process may be implemented in software executed on surgical computer 150 shown in FIG. 1.

In the preoperative phase 410, the modules, for example, 302, 304, 306, are prepared and calibrated in accordance with calibration procedure already implemented by the software of CASS 100. At 412, the software enters calibration mode and at 413, the software requests calibration of the first module. The module is calibrated at 414 and, at 415, the calibration information is stored, preferably within CASS 100. The calibration process may comprise, for example, touching the tip of the tool 310 at a predetermined spot such that the position of the fiducial markers 202 attached to the handheld device 105B are visible to the tracking system 115. The software may provide instructions to the surgeon as to the exact steps need to calibrate each module and may further provide a verification of the successful completion of the calibration. At 416, it is determined whether all modules which been prepared in anticipation of the surgical procedure have been calibrated. For example, the software may inquire to the surgeon if there are more modules requiring calibration. If there are more modules, control returns to box 413 where the software may request that the surgeon calibrate the next module. The process is repeated until, at 416, all modules have been calibrated, at which time the calibration process exits at 417.

In the intra-operative phase 420, the surgeon begins a surgical procedure at 421. The surgical procedure may require the use of the handheld device 105B having one of modules 302, 304, 306 attached thereto. At 422, the software waits for an indication that the surgery has been completed and, if no such indication is given, at 423 the software determines if a tool change request has been received. If no tool change request has been received at 423, the software returns to decision point 422 to determine if the surgery has been completed. When a tool change request has been received at 423, the software may prompt the surgeon to provide an indication of which tool will be used next and, in addition, may provide instructions to the surgeon to connect the selected module to the handheld device 105B. At this time, at 424, the calibration information from the pre-operative calibration procedure 410 is loaded and the surgeon is thereafter free to use the newly-selected tool. Control returns to loop 422-423 where the process waits for completion of the surgery or for an additional tool change request. If, at decision point 422, the system software receives an indication that surgery is completed, the surgical procedure is ended at 425.

In a second example of the present disclosure, the fixation mechanism 204 is provided with a sensor that conveys to the software of CASS 100 the extent of the insertion of the shaft 207 of surgical tool 206 into the fixation mechanism 204. Preferably, the sensor will be calibrated such that the software can determine the position of the tip of tool 206 based on readings from the sensor and can indicate to the surgeon when to stop the insertion. When the shaft 207 of tool 206 has been inserted the required distance into the fixation mechanism 204, the software will provide an indication to the surgeon to stop the insertion.

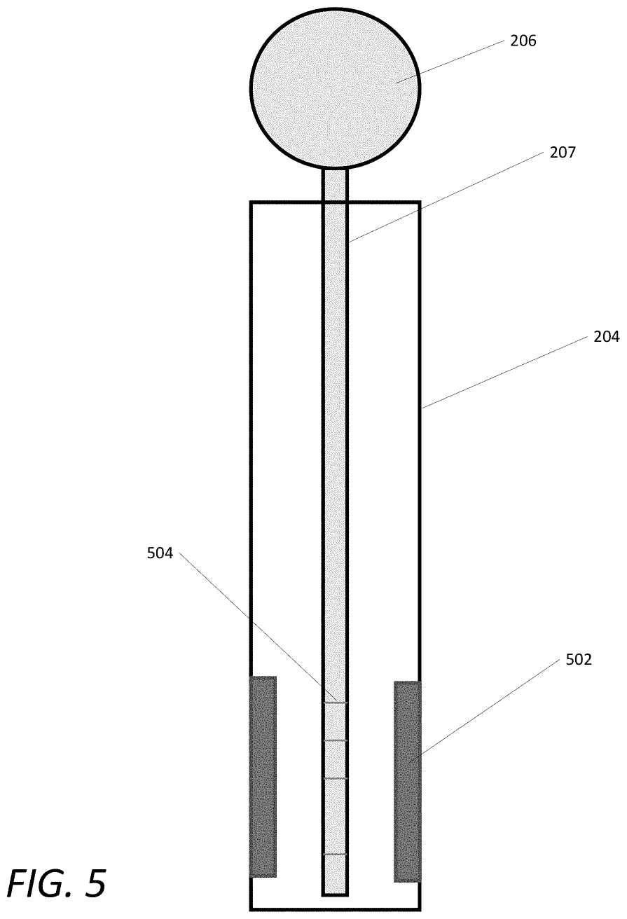

In the first variation of the second example, shown in FIG. 5, fixation mechanism 204 is provided with an optical sensor 502 which is capable of sensing one or more markings 504 on the shaft portion 207 of tool 206. Based on the number and position of the markings 504 on shaft 207, as detected by optical sensor 502, the software can determine the degree to which tool 206 has been inserted into fixation mechanism 204. Based on the sensed position of the shaft portion 207 of tool 206, the software can provide instructions to the surgeon as to whether to further insert tool 206 into fixation mechanism 204 or to retracted tool 206 from fixation mechanism 204, until the proper positioning of tool 206 within fixation mechanism 204 is achieved. Once the proper positioning of tool 206 has been achieved, the software can signal the surgeon to secure fixation mechanism 204 around the shaft 207 of tool 206. As such, the software, based on the geometry of tool 206, knows the exact position of the tip of tool 206 with respect to fixation mechanism 204, handheld device 105B, and the array of fiducial markers 202 attached to handheld device 105B. As such, when handheld device 105B is moved by the surgeon, the position of the tip of tool 206 may easily be determined.

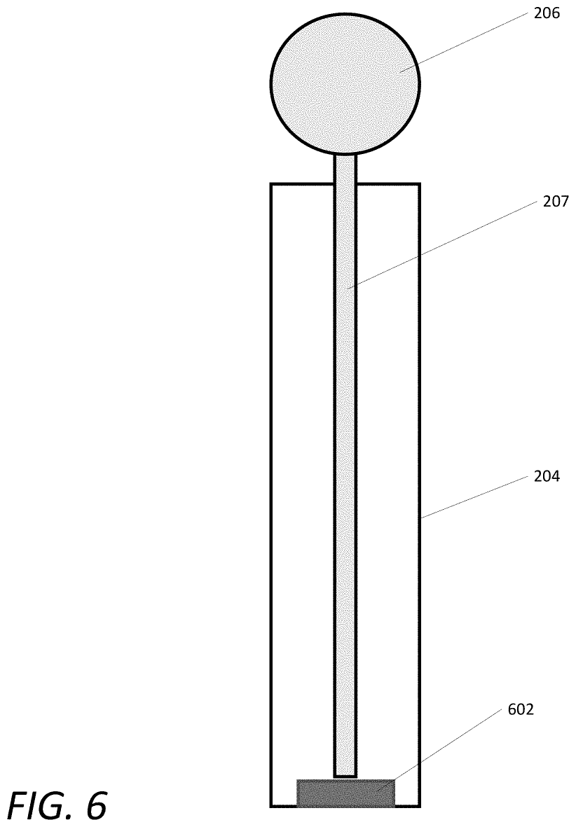

In a second variation of the second example, shown in FIG. 6, a pressure sensor 602, which may sense pressure thereon produced by the contact of shaft 207 of tool 206 with pressure sensor 602 when the tool 206 is inserted into fixation mechanism 204. As with the first variation of the second example, the software may determine, based on the detected pressure, when the tool 206 is in the correct position within fixation mechanism 204 and may instruct the surgeon to stop the insertion of tool 206.

It should be noted that, in other variations of the second example, various other types of sensors may be used determine the exact position of tool 206 and, in particular, the shaft 207 of tool 206, within fixation mechanism 204.

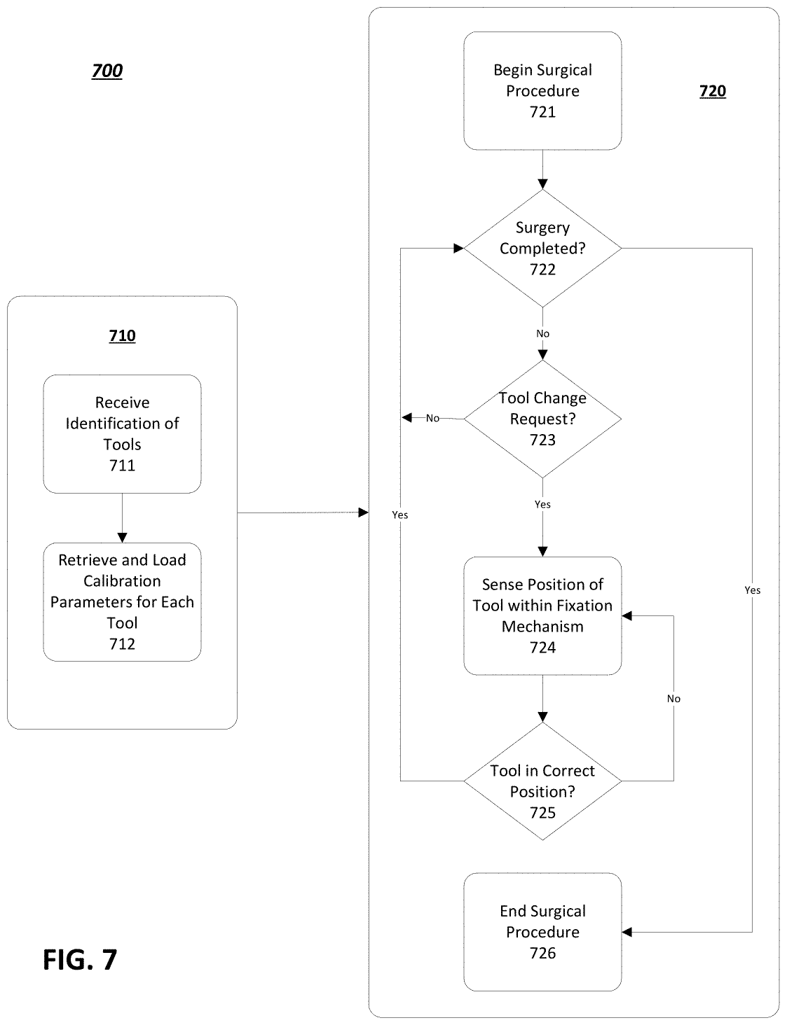

FIG. 7 is a flowchart showing a process 700 implemented as part of the software of CASS 100 for supporting the second example of the present disclosure. In one example, in a pre-operative phase 710, the system software receives an identification of the tools to be used during the surgical procedure from the surgeon and, at 712, retrieves and loads the calibration parameters for each tool 206. The calibration parameters may comprise, at least, sensor readings to be received from sensor (502 or 602) within fixation mechanism 204 which indicates proper positioning of tool 206. In alternative examples, the steps indicated in the pre-operative phase 710 may be performed intra-operatively.

In the intra-operative phase 720, the surgeon begins a surgical procedure at 721. The surgical procedure may require the use of the handheld device 105B having a tool 206 attached thereto. At 722, the software waits for an indication that the surgery has been the completed and, if no such indication is given, at 723, determines if a tool change request has been received. If no tool change request has been received at 723, the software returns to decision point 722 to determine if the surgery has been completed. When a tool change request has been received at 723, the software may prompt the surgeon to provide an indication of which tool will be used next and, in addition, will provide feedback to the surgeon as to how far to insert the shaft 207 of tool 206 into or retract (in the case for the surgeon has inserted the tool too far) the shaft 207 from fixation mechanism 204. The feedback is based on the expected readings from the sensor (502, 602) associated with the tools 206 that were identified in pre-operative phase 710 or during intraoperative phase 720. At decision point 725, it is determined if the tool 206 is in the correct position within fixation mechanism 204, and, if not, control returns to 724 where the position of the tool 206 within the fixation mechanism 204 is further sensed. If, at 725, the tool is in the correct position, the surgeon may be instructed to secure the tool 206 within fixation mechanism 204 and is thereafter free to use the newly-selected tool. Control returns to loop 722-723 where the process waits for completion of the surgery or for an additional tool change request. If, at decision point 722, the system software receives an indication that surgery is completed, the surgical procedure is ended at 726.

In alternative examples, other methods may be used to localize the surgical tool with respect to the fixation mechanism. For example, U.S. Pat. No. 9,031,637 discloses three methods for determining the depth of a drill bit placement within a hand-held tool. Any of these mechanisms specified therein for use with the drill bit could be adapted for use herein to localize the surgical tool with respect to the fixation mechanism.

In one alternative example, a drill bit may act as a stator moving relative to a slider, wherein the stator and the slider form a capacitive array that can sense relative motion. By moving the stater and slider in linear relation relative to one another, a voltage fluctuation occurs that can be interpreted and used to determine the travel distance of the drill bit.

In a second alternative example, the system may include a reflective code strip having alternating light and dark areas defined thereon, a lens and an encoder. As the code strip rotates, light focused by the lens creates an alternating pattern of light and shadow which is sensed by the encoder. The encoder converts the pattern into a digital output representing linear motion of the drill bit.

In yet a third alternative example, a linear variable differential transformer is used. The transformer includes a plurality of solenoid cells placed end-to-end around the tube to. A cylindrical ferromagnetic core, for example, the drill bit, slides along the axis of the tube, thereby driving an alternating current through a primary coil causing a voltage to be induced in each secondary of the transformer proportional to its mutual inductance with the primary. The magnitude of the induced voltage indicates the distance moved by the drill bit and the phase of the voltage indicates the direction of the displacement.

In the above detailed description, reference is made to the accompanying drawings, which form a part hereof. In the drawings, similar symbols typically identify similar components, unless context dictates otherwise. The illustrative examples described in the present disclosure are not meant to be limiting. Other examples may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular examples described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular examples only and is not intended to be limiting.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those of skill in the art that virtually any disjunctive word and/or phrase presenting two or more alternative terms, whether in the description, sample examples, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those of skill in the art, each of which is also intended to be encompassed by the disclosed examples.

1. A method comprising:
for each of one or more surgical tools:
placing the surgical tool in a fixation mechanism;
attaching the fixation mechanism to a handheld device to create a surgical tool/fixation mechanism combination;
calibrating the handheld device with the fixation mechanism and surgical tool; and
storing the calibration information for the tool.

2. The method of claim 1, further comprising:
removing the surgical tool/fixation mechanism combination from the handheld device.

3. The method of claim 1, wherein the step of calibrating comprises:
touching the surgical tool to one or more predefined spots such that a position of fiducial markers attached to the handheld device may be read by a tracking system.

4. A system comprising:
a processor; and
software that, when executed by the processor, causes the system to perform the method of claim 2.

5. The system of claim 4, wherein the software further causes the system to:
receive an identification of one or more surgical tools to be used during a surgical procedure; and
instruct a user to serially calibrate each of the one or more surgical tools.

6. The system of claim 5, wherein the software further causes the system to:
provide instructions to the user indicating steps required to calibrate each surgical tool.

7. The system of claim 5, wherein the software, in a preoperative phase, further causes the system to:
identify a calibration procedure for the identified surgical tools; and
calibrate each identified surgical tool using the identified procedures.

8. The system of claim 7, wherein the software implements calibration procedures for the one or more surgical tools.

9. The system of claim 4, wherein the software further causes the system to:
receive an interoperative request to change the surgical tool; and
prompt a user to identify the next desired surgical tool.

10. The system of claim 9, wherein the software further causes the system to:
retrieve and load calibration information for the requested surgical tool.

11. The system of claim 10, wherein the software further causes the system to:
instruct the user to connect the requested surgical tool and fixation mechanism to the handheld device.

12. A device comprising:
a powered handheld device;
a fixation mechanism, removably attached to the handheld device; and
a surgical tool held by the fixation mechanism;
wherein one or more combination surgical tools/fixation mechanisms are preoperatively attached to the handheld device and calibrated.

13. The device of claim 12, wherein the combination surgical tool/fixation mechanism attached to the powered handheld device may be dynamically changed during a surgical procedure by disconnecting a first fixation mechanism containing a first surgical tool and connecting a second fixation mechanism containing a second surgical tool to the powered handheld device.

14. A device comprising:
a powered handheld device;
a fixation mechanism, removably attached to the handheld device;
a surgical tool held by the fixation mechanism; and
a sensor for sensing an extent to which a shaft of the surgical tool has been inserted into the fixation mechanism.

15. The device of claim 14, wherein the sensor is an optical sensor and further wherein the shaft of the surgical tool has markings thereon detectable by the optical sensor.

16. The device of claim 14, wherein the sensor is a pressure sensor sensing pressure exerted by the shaft of the surgical tool as it is inserted into the fixation mechanism.

17. A system comprising:
a processor; and
software that, when executed by the processor, causes the system to:
receive an interoperative request to change the surgical tool; and
retrieve and load calibration information for the requested surgical tool.

18. The system of claim 17, wherein the software causes further causes the system to:
receive preoperative identification of all surgical tools to be used during a surgical procedure; and
store calibration parameters associated with the identified surgical tools.

19. The system of claim 17, wherein the software causes further causes the system to:
prompt a user to identify the next desired surgical tool.

20. The system of claim 19, wherein the software causes further causes the system to:
provide feedback to a user indicating proper positioning of the shaft of the surgical tool with respect to the fixation mechanism, based on the calibration parameters and readings from the sensor.
